(19) United States
(12) Patent Application Publication
(10) Pub. No.: US 2024/0225891 A1
(43) Pub. Date: Jul. 11, 2024

(54) Flexible Ice Container for helping heal injuries to human body parts

(71) Applicant: John Christian Benneche, Newbury Park, CA (US)

(72) Inventor: John Christian Benneche, Newbury Park, CA (US)

(21) Appl. No.: 18/152,911

(22) Filed: Jan. 11, 2023

(51) Int. Cl. A61F 7/10

(52) U.S. Cl. CPC A61F 7/103; A61F 2007/108

(57) ABSTRACT

An ice massage therapy device making use of exposed ice within a hand-held flexible container that folds down onto itself exposing the ice where the internal ice is held in place with protrusions on the inner wall.

### STATEMENT REGARDING FEDERALLY SPONSORED RESEARCH OR DEVELOPMENT

N/A

### THE NAMES OF THE PARTIES TO A JOINT RESEARCH AGREEMENT

N/A

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

N/A

### STATEMENT REGARDING PRIOR DISCLOSURES BY THE INVENTOR OR A JOINT INVENTOR

N/A

### BACKGROUND OF THE INVENTION

### (1) Field of the Invention

The present application relates to a device that is used in the field of Physical Therapy using exposed ice elements for helping heal injury to a human body.

### (2) Description of Related Art Including Information Disclosed Under 37 CFR 1.97 and 1.98

It has long been known that ice is used to help with pain management and healing human tissue. One need only search the internet for the relevant topic and it's clear that icing is a main component to healing injuries. A trip to a physical therapist or a chiropractor would yield a probable ice treatment at the end of your visit. The proliferation of commercial cryo chambers for “freezing” therapies furthers this claim.

Ice Packs abound in your local pharmacy as well. One can buy them at the store or on the internet in many shapes and sizes. At home, ice therapy has been around for a very long time.

U.S. Pat. No. 7,065,983 illustrates an ice bag used to help heal injuries. U.S. Pat. No. 5,065,758 shows another device for using cold temperatures to promote healing. Same for U.S. Pat. No. 4,628,932 and their knee ice pack. U.S. Pat. No. 6,945,988 shows a device for cooling shoulder joints and nearby muscles. U.S. Pat. No. 4,044,773 is another cold therapy package. These all make use of cold to help heal as does this device in this patent application. However, none make use of exposing ice to the skin nor are they refillable or made from flexible material that is used to fold over for exposing ice for contact directly to the skin nor for massage like activity.

U.S. Pat. No. 7,112,178 shows a device used as a cold therapy foot massager. U.S. Pat. No. 6,129,687 shows yet another hot-cold roller used for therapy. U.S. Pat. No. 4,884,560 shows another thermal massage device. Application US 2015/0057579 A1 shows a freezable rolling massage device. While these all address healing from a cold therapy with massage-like action, they differ in that none of them are made from a flexible material that folds over and reshapes to expose ice and cover ice for refill for use.

As can be seen, freezing liquids and using them for injury therapy is nowhere close to a novel idea. The act of using frozen water, ice directly onto the skin is not novel either. U.S. Pat. No. 6,953,459B2, an ice dispenser for cold therapy is a close approximation to this new idea by having exposed frozen water, ice elements, applied directly to the skin yet the means to achieve contact with the skin is very different as this new idea makes use of folding flexible material and does not make use of directional controller or aperture nor is the main body of U.S. Pat. No. 6,953,459B2 made of flexible material.

### BRIEF SUMMARY OF THE INVENTION

An improved method of helping heal the human body with frozen ice in a container. The hand-held container made of non-porous flexible material is filled with water then frozen and once frozen the walls of the container are folded down onto themselves exposing the encased ice to be used for applying to human skin for healing. The container's inside wall is constructed to inhibit the ejecting of the ice once it is in use.

### BRIEF DESCRIPTION OF THE SEVERAL VIEWS OF THE DRAWING(S)

FIG. 1 shows the invention in an empty state. Container is shown with opening at 10, sidewall 11 of the container, and indentation 12 that produces a protrusion on the inside wall.

FIG. 2 shows a cutaway view of the container with opening 10 at the top, with the sidewall 11, indentation 12 on outer sidewall, and internal protrusion 13. Closed distal end 14 is shown on the bottom

FIG. 3 Shows the container in ready to use form with frozen ice 15 exposed from having folded sidewall 11 down and onto itself.

### DETAILED DESCRIPTION OF THE INVENTION

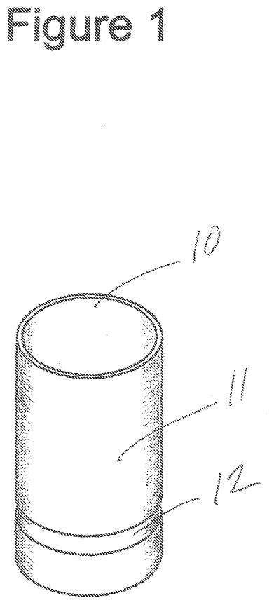

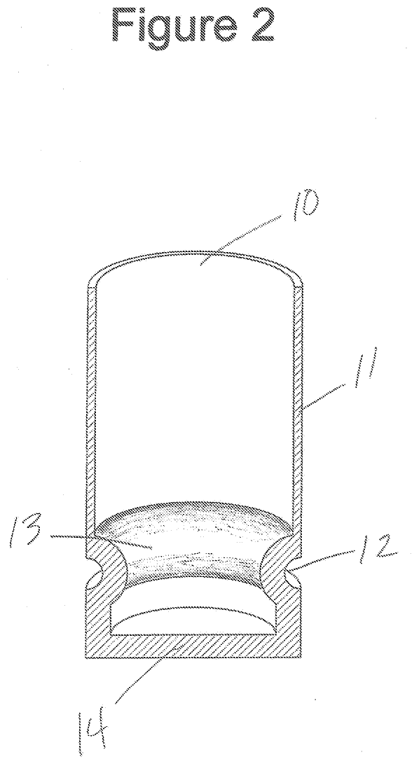

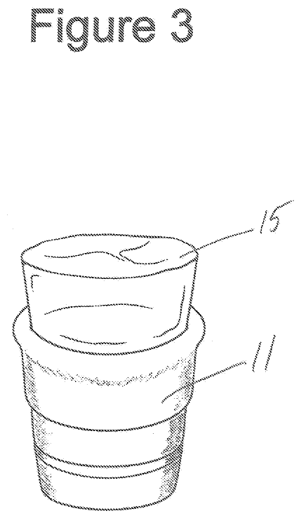

A user would pour liquid through opening 10 in FIG. 1 and place it in the freezer. Said liquid is contained by bottom barrier 14 and sidewall 11 in FIG. 2. Once the liquid is frozen and the container removed from the freezer, the user of the container would stretch and fold over sidewall 11 onto itself exposing ice 15 for use as shown in FIG. 3. Internal protrusion 13 as shown in FIG. 2 would keep the frozen ice from falling out while in use. The frozen ice surrounds internal protrusion 13 effecting this feature. After use, one refolds sidewall 11 up to original shape and places it back in the freezer for further use or refills with liquid and then freezes for further use.

1. An ice therapy container comprising:
an opening for filling with liquid on one end and closed at the opposite end with containing walls in between the open end and closed end for trapping liquid for freezing;
an internal wall with at least one protrusion for securing frozen liquid in its place;
construction material that will expand from the forces of liquid turning to solid; and
walls that can be folded down onto themselves and back up to original position

2. The ice therapy container of claim 1 in wherein said container is constructed from non-porous material.

3. The ice therapy container of claim 1 in wherein said container is constructed from material that can be stretched such as rubber or silicone rubber

4. The ice therapy container of claim 1 wherein said container has the ability to contain liquid until frozen.

6. The ice therapy container of claim 1 wherein said container can be held by hand
